(19) United States
(12) Patent Application Publication
(10) Pub. No.: US 2025/0072845 A1
(43) Pub. Date: Mar. 6, 2025

(54) PET SYSTEM AND CORRECTION METHOD AND SYSTEM THEREOF, DEVICE, AND MEDIUM

(71) Applicant: Shanghai United Imaging Healthcare Co., Ltd., Shanghai (CN)

(72) Inventors: Liuchun He, Shanghai (CN); Yun Dong, Shanghai (CN)

(21) Appl. No.: 18/820,256

(22) Filed: Aug. 30, 2024

(30) Foreign Application Priority Data
Aug. 30, 2023 (CN) 202311110336.9

(51) Int. Cl. A61B 6/03; A61B 6/42; G06T 11/00

(52) U.S. Cl. CPC A61B 6/037; A61B 6/4266; G06T 11/005

(57) ABSTRACT

The present disclosure provides a PET system and a correction method and system thereof, a device, and a medium. The correction method includes: obtaining an initial NC factor of an initial LOR corresponding to a pair of coincident detector crystals in the PET system; determining, based on the DOI information, a target LOR corresponding to the pair of coincident detector crystals; obtaining a DOI compensating coefficient for the NC factor of the target LOR relative to the initial LOR; and obtaining, based on the initial NC factor and the DOI compensating coefficient for the NC factor, a target NC factor corresponding to the target LOR to correct the PET system.

### CROSS-REFERENCE TO RELATED APPLICATIONS

This application claims priority to Chinese patent application No. 202311110336.9, filed on Aug. 30, 2023, the content of which is hereby incorporated by reference in its entirety.

### TECHNICAL FIELD

The present disclosure relates to the field of positron emission tomography (PET) technologies, and in particular, to a PET system and a correction method and system thereof, a device, and a medium.

### BACKGROUND

PET is a high-end nuclear medicine imaging diagnostic technology. In actual operation, a radioactive nuclide is used to label a metabolite, and this labeled metabolite will emit a positron through decay emission, the positron annihilates with a surrounding electron to produce a pair of photons emitted in opposite directions. If the two photons are detected simultaneously by detector crystals of a PET detection system, then the nuclide is considered to be on a line connecting the corresponding pair of detectors (a group of detectors) that has captured the photons, and this line is called a line of response (LOR). The collection of all the LORs in each inspection constitutes raw data of PET, which is called list-mode data. When using PET to perform large-scale imaging, a mobile scanning mode is generally used, and a moving position of a bed is generally transmitted to the PET detection system in real time and stored in the list-mode data. By performing a series of physical corrections on the list-mode data, a three-dimensional image for showing the functional metabolism and reflecting the metabolic activities of life is finally generated by using a reconstruction algorithm.

Since the depth at which annihilation photons interact with the detector crystals is arbitrary and even inter-crystalline penetration may occur, there is a severe radial depth of interaction (DOI) effect or radial parallax in a certain lateral field of view of the PET system, resulting in uneven resolution of a reconstructed image at different radial positions. In particular, when an object is close to an edge of the field of view, the resolution will be significantly reduced, resulting in a serious “tailing” effect.

The common solutions for normalization correction (NC) of the PET system are direct normalization or component normalization. The direct normalization is generally used in two-dimensional (2D) PET systems with fewer LORs. For the three-dimensional (3D) PET systems, it is generally impracticable because the required statistics are too large.

The component normalization is a method that decomposes the normalization in the level of LOR into multiple components in smaller dimensions, thereby reducing the complexity of correction and the required statistics. For example, a correction method by moving a phantom is used for long-axis systems. However, these methods currently do not involve PET systems with DOI information.

The DOI information specifically refers to the depth information of the deposition of gamma rays in the PET detector, which facilitates improving the spatial resolution of the PET system. However, since the detector with DOI information is divided from an original single crystal structure into multiple crystal layers with different depths, the number of LORs of the PET system is greatly increased. Assuming that the PET system has 8 layers of DOI, the number of LORs is 82 times that of the PET system without DOI information. The large increase in LOR also significantly increases the difficulty of the NC.

Assuming that the number of DOI layers is relatively small, it is worth considering the use of the conventional component normalization method, by simply doubling the number of the crystal layers of the detector. However, for the detector with a large number of layers of DOI, this method cannot be used anymore, or is extremely difficult to use due to the huge required statistics.

### SUMMARY

The technical problem to be solved by the present disclosure is to provide a PET system and a correction method and system thereof, a device, and a medium in order to overcome the defect in the conventional art that there is a lack of NC for detectors with DOI information.

The present disclosure solves the above technical problems through the following technical solutions.

The positive and progressive effects of the present disclosure are as follows.

In a first aspect, a correction method for a PET system is provided, which is applied to a PET system including DOI information (DOI interlayer information). The correction method includes:
obtaining an initial NC factor of an initial LOR corresponding to a pair of coincident detector crystals in the PET system; the initial LOR being unrelated to the DOI information;
determining, based on the DOI information, a target LOR corresponding to the pair of coincident detector crystals;
obtaining a DOI compensating coefficient for the NC factor of the target LOR relative to the initial LOR; and
obtaining, based on the initial NC factor and the DOI compensating coefficient for the NC factor, a target NC factor corresponding to the target LOR to correct the PET system.

In an embodiment, the step of obtaining the initial NC factor of the initial LOR corresponding to the pair of coincident detector crystals in the PET system includes:
obtaining, based on a preset NC method, the initial NC factor of the initial LOR corresponding to the pair of coincident detector crystals in the PET system;
the preset NC method including a direct NC method and/or a component NC method.

In an embodiment, the step of obtaining, based on the initial NC factor and the DOI compensating coefficient for the NC factor, the target NC factor corresponding to the target LOR to correct the PET system includes:
determining a product of the initial NC factor and the DOI compensating coefficient for the NC factor, and using the product as the target NC factor to correct the PET system.

In an embodiment, the step of obtaining the DOI compensating coefficient for the NC factor of the target LOR relative to the initial LOR includes:
obtaining a total count of initial LORs unrelated to the DOI information corresponding to the pair of coincident detector crystals;
obtaining a total count of target LORs related to the DOI information corresponding to the pair of coincident detector crystals; and
obtaining, based on the total count of the initial LORs and the total count of the target LORs, the DOI compensating coefficient for the NC factor of the target LOR relative to the initial LOR.

In an embodiment, the step of obtaining, based on the total count of the initial LORs and the total count of the target LORs, the DOI compensating coefficient for the NC factor of the target LOR relative to the initial LOR includes:
calculating a ratio of the total count of the initial LORs to the total count of the target LORs, and using the ratio as the DOI compensating coefficient for the NC factor.

In an embodiment, the correction method further includes:
obtaining a spatial position corresponding to each target LOR;
merging a plurality of target LORs located within a same preset spatial position range into a same virtual reconstructed LOR;
obtaining, based on target NC factors corresponding to the plurality of target LORS, respectively, a reconstructed NC factor corresponding to the virtual reconstructed LOR; and
correcting, based on the reconstructed NC factor, the PET system;
and/or the step of obtaining, based on the initial NC factor and the DOI compensating coefficient for the NC factor, the target NC factor corresponding to the target LOR to correct the PET system includes:
obtaining, based on the initial NC factor and the DOI compensating coefficient for the NC factor, the target NC factor corresponding to the target LOR; and
reconstructing, based on the target NC factor, a PET image to correct the PET system.

In a second aspect, a correction system for a PET system is further provided, which is applied to a PET system including DOI information. The correction system includes:
a first data obtaining module configured to obtain an initial NC factor of an initial LOR corresponding to a pair of coincident detector crystals in the PET system; the initial LOR being unrelated to the DOI information;
a second data obtaining module configured to determine, based on the DOI information, a target LOR corresponding to the pair of coincident detector crystals;
a third data obtaining module configured to obtain a DOI compensating coefficient for the NC factor of the target LOR relative to the initial LOR; and
a first data processing module configured to obtain, based on the initial NC factor and the DOI compensating coefficient for the NC factor, a target NC factor corresponding to the target LOR to correct the PET system.

In an embodiment, the first data obtaining module is further configured to obtain, based on a preset NC method, the initial NC factor of the initial LOR corresponding to the pair of coincident detector crystals in the PET system.

The present NC method includes a direct NC method and/or a component NC method.

In an embodiment the first data processing module is further configured to determine a product of the initial NC factor and the DOI compensating coefficient for the NC factor, and use the product as the target NC factor to correct the PET system.

In an embodiment, the third data obtaining module includes:
an initial total count obtaining unit configured to obtain a total count of initial LORs unrelated to the DOI information corresponding to the pair of coincident detector crystals;
a target total count obtaining unit configured to obtain a total count of target LORs related to the DOI information corresponding to the pair of coincident detector crystals; and
a correction coefficient obtaining unit configured to obtain, based on the total count of the initial LORs and the total count of the target LORs, the DOI compensating coefficient for the NC factor of the target LOR relative to the initial LOR.

In an embodiment, the correction coefficient obtaining unit is further configured to calculate a ratio of the total count of the initial LORs to the total count of the target LORs, and use the ratio as the DOI compensating coefficient for the NC factor.

In an embodiment, the correction system further includes:
a position obtaining module configured to obtain a spatial position corresponding to each target LOR;
a merging module configured to merge a plurality of target LORs located within the same preset spatial position range into a same virtual reconstructed LOR;
a reconstruction factor obtaining module configured to obtain, based on target NC factors corresponding to the plurality of target LORs, respectively, a reconstructed NC factor corresponding to the virtual reconstructed LOR; and
a correction module configured to correct, based on the reconstructed NC factor, the PET system;
and/or the first data processing module includes:
a target factor obtaining unit configured to obtain, based on the initial NC factor and the DOI compensating coefficient for the NC factor, the target NC factor corresponding to the target LOR; and
an image reconstruction unit configured to reconstruct, based on the target NC factor, a PET image to correct the PET system.

In a third aspect, a PET system is further provided, including the above correction system for a PET system.

In a fourth aspect, an electronic device is further provided. The electronic device includes a memory, a processor, and a computer program stored on the memory and configured to run on the processor. When the processor executes the computer program, the above correction method for a PET system is implemented.

In a fifth aspect, a computer-readable storage medium having a computer program stored therein is further provided. When the computer program is executed by a processor, the above correction method for a PET system is implemented.

On the basis of conforming to the common sense in the art, the above-mentioned features of various embodiments can be arbitrarily combined, and these combinations should all fall within the scope of the present disclosure.

In the PET system and the correction method and system thereof, the device, and the medium of the present disclosure, based on the traditional initial NC factor of the initial LOR unrelated to the DOI information, the DOI compensating coefficient for the NC factor of the target LOR related to the DOI information relative to the initial LOR is introduced. Then the target NC factor corresponding to the target LOR is obtained through the initial NC factor and the DOI compensating coefficient for the NC factor, thereby achieving the NC for the PET system. Without increasing the complexity and statistics of the correction, the accuracy of the reconstruction of image by the detector in the PET system with the DOI information is improved, and the maintainability of the PET system is significantly improved.

### BRIEF DESCRIPTION OF THE DRAWINGS

FIG. 1 is a first flow diagram illustrating a correction method for a PET system according to Embodiment One of the present disclosure.

FIG. 2 is a second flow diagram illustrating the correction method for a PET system according to Embodiment One of the present disclosure.

FIG. 3 is a third flow diagram illustrating the correction method for a PET system according to Embodiment One of the present disclosure.

FIG. 4 is a schematic diagram illustrating a target LOR in the correction method for a PET system according to Embodiment One of the present disclosure.

FIG. 5 is a fourth flow diagram illustrating the correction method for a PET system according to Embodiment One of the present disclosure.

FIG. 6 is a fifth flow diagram illustrating the correction method for a PET system according to Embodiment One of the present disclosure.

FIG. 7 is a sixth flow diagram illustrating the correction method for a PET system according to Embodiment One of the present disclosure.

FIG. 8 is a seventh flow diagram illustrating the correction method for a PET system according to Embodiment One of the present disclosure.

FIG. 9 is a schematic diagram illustrating a reconstructed image of the correction method for a PET system according to Embodiment One of the present disclosure.

FIG. 10 is a schematic diagram illustrating a configuration of a correction system for a PET system according to Embodiment Two of the present disclosure.

FIG. 11 is a schematic diagram illustrating a configuration of a PET system according to Embodiment Three of the present disclosure.

FIG. 12 is a schematic diagram illustrating a configuration of an electronic device according to Embodiment Four of the present disclosure.

### DETAILED DESCRIPTION OF THE EMBODIMENTS

The present disclosure is further described below by way of embodiments, but the present disclosure is not limited to the scope of the embodiments.

### Embodiment One

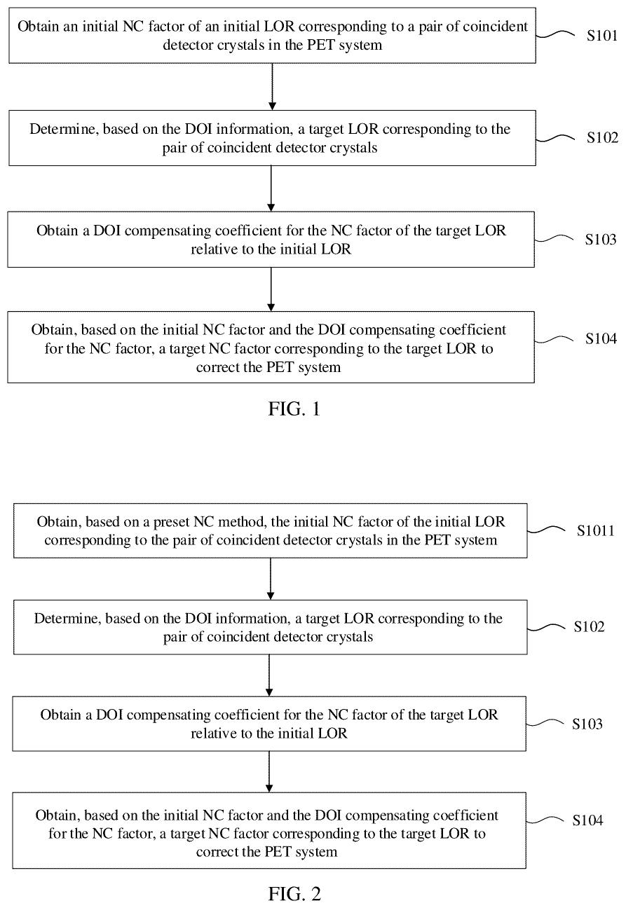

The present embodiment provides a correction method for a PET system, which is applied to a PET system including DOI information. As shown in FIG. 1, the correction method includes the following steps.

In step S101, an initial NC factor of an initial line of response (LOR) corresponding to a pair of coincident detector crystals in the PET system is obtained.

The initial LOR is unrelated to the DOI information.

In step S102, a target LOR corresponding to the pair of coincident detector crystals is determined based on the DOI information.

The target LOR is related to the DOI information.

In step S103, a DOI compensating coefficient for the NC factor of the target LOR relative to the initial LOR is obtained.

In step S104, based on the initial NC factor and the DOI compensating coefficient for the NC factor, a target NC factor corresponding to the target LOR is obtained to correct the PET system.

It is to be noted that, unless otherwise defined or a conflict appears, the performing sequences of the steps in the embodiments of the present disclosure are not limited. For example, the above step S101 may be performed before or after the step S102, or the steps S101 and S102 may be performed concurrently.

For example, the correction method may alternatively include the following steps.

In a first step, a target LOR corresponding to a pair of coincident detector crystals in the PET system is determined based on the DOI information.

The target LOR is related to the DOI information.

In a second step, an initial NC factor of an initial LOR corresponding to the pair of coincident detector crystals is obtained.

The initial LOR is unrelated to the DOI information.

In a third step, a DOI compensating coefficient for the NC factor of the target LOR relative to the initial LOR is obtained.

In a fourth step, based on the initial NC factor and the DOI compensating coefficient for the NC factor, a target NC factor corresponding to the target LOR is obtained to correct the PET system.

For a PET system with a certain number of rings, each of the rings has a certain number of detector crystals, and a coincidence event corresponds to two detector crystals. The two detector crystals corresponding to a certain coincidence event constitute a group of coincident detector crystals (i.e., a target detector crystal group) in the present disclosure, and may also be referred to as a pair of coincident detector crystals (i.e., a target detector crystal pair).

The initial LOR corresponding to the pair of coincident detector crystals in the PET system is a LOR without depth of interaction (DOI) information, and the target LOR corresponding to the pair of coincident detector crystals is a LOR with the DOI information.

For a PET system including the DOI information, its detector crystal has certain DOI information, i.e., the crystal has a certain number of layers. Based on the DOI information, it is not only possible to determine the pair of coincident detector crystals where the coincidence event occurs, but also possible to determine a layer of the crystal where the coincidence event occurs, i.e., the depth information. For example, assuming that a detector crystal has 8 layers, and a coincidence event occurred in the 5th layer of one crystal and the 6th layer of the other crystal, corresponding to a depth of 1 cm and a depth of 1.5 cm from the surface of the crystal, respectively, then based on the DOI information, the LOR of the coincidence event can be determined more accurate, thereby improving the resolution of the reconstructed image.

In the correction method for a PET system of the present embodiment, based on the traditional initial NC factor of the initial LOR unrelated to the DOI information, the DOI compensating coefficient for the NC factor (i.e. a normalization factor correction coefficient) of the target LOR related to the DOI information relative to the initial LOR is introduced. Then the target NC factor corresponding to the target LOR is obtained through the initial NC factor and the DOI compensating coefficient for the NC factor, thereby achieving the NC for the PET system. Without increasing the complexity and statistics of the correction, the accuracy of the reconstruction of image by the detector in the PET system with the DOI information is improved, and the maintainability of the PET system is significantly improved.

In some embodiments, as shown in FIG. 2, the above step S101 includes the following step.

In step S1011, based on a preset NC method, the initial NC factor of the initial LOR corresponding to the pair of coincident detector crystals in the PET system is obtained.

The preset NC method includes a direct NC method and/or a component NC method.

The initial LOR corresponding to the pair of coincident detector crystals in the PET system is a LOR without the DOI information, and the initial NC factor corresponding to the initial LOR is unrelated to the DOI information. How to obtain the initial NC factor of the initial LOR is a prior art, and will not be repeated here.

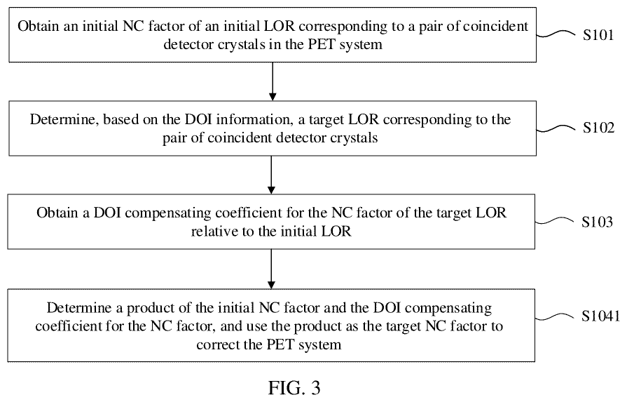

In some embodiments, as shown in FIG. 3, the above step S104 includes the following step.

In step S1041, a product of the initial NC factor and the DOI compensating coefficient for the NC factor is determined, and the product is used as the target NC factor to correct the PET system.

For example, an initial NC factor is represented by Nc, a DOI compensating coefficient for the NC factor is represented by D, and a target NC factor is represented by Nm. Then, the target NC factor is the product of the initial NC factor and the DOI compensating coefficient for the NC factor, i.e., Nm=Nc*D.

A specific example is given below to further illustrate this implementation.

Specifically, for a traditional cylindrical PET system with a specific number of rings, the initial NC factor of the initial LOR of the coincidence event between the i^{th} crystal a in the u^{th} ring and the j^{th} crystal b in v^{th} the ring is represented as Nc=N(ui,vj), where u, i, v, and j are each an integer, u and v are each the ordinal number of a ring where the crystal locates, and u and v can each have a value within a range of the total number of rings in the system; and i and j are each the ordinal number of the crystal, and i and j can each have a value within a range of the total number of crystals in a single ring. That is, ui corresponds to the crystal a, and vj corresponds to the crystal b. The crystal a and the crystal b constitute a pair of coincident detector crystals, a line connected between a photon incident point of the crystal a and a photon incident point of the crystal b is equivalent to an initial LOR, and the initial NC factor of the initial LOR is represented as Nc=N(ui,vj).

If the DOI information corresponds to the m^{th} layer in the crystal a and the n^{th} layer in the crystal b, it can be determined that the target LOR occurred in the m^{th} layer in the crystal a and the n^{th} layer in the crystal b, then the target NC factor corresponding to the target LOR is represented as Nm=N(uim,vjn), where m and n are each a positive number, and m and n are each the ordinal number of a DOI layer where the target LOR occurred, and m and n can each have a value within a range of the total number of DOI layers of a single crystal in the PET system. That is, uim corresponds to the m^{th} layer in the crystal a, and vjn corresponds to the n^{th} layer in the crystal b. A line connected between a photon incident point of the m^{th} layer of the crystal a and a photon incident point of the n^{th} layer of the crystal b is a target LOR. The target LOR is a more accurate LOR than the initial LOR, and the target NC factor of the target LOR is represented as Nm=N(uim,vjn).

The DOI compensating coefficient for the NC factor of the target LOR relative to the initial LOR, can be determined based on one or more parameters. In an exemplary embodiment, the one or more parameters may include one or more of: DOI information, crystal ring difference, radial index of the target LOR (which may also be called as radial ordinal number of the target LOR), or transaxial angular index of the target LOR (which may also called as radial angle ordinal number of the target LOR). It should be noted, the crystal ring difference, the radial index, and the transaxial angular index of the target LOR are parameters used to present the position of the target LOR, which are non-limiting. The position of the target LOR can be presented in other manners, e.g., space coordinate, etc. Therefore, in other embodiments, the DOI compensating coefficient for the NC factor may be represented by other parameters.

Exemplarily, the DOI compensating coefficient for the NC factor of the target LOR relative to the initial LOR may be represented as D=D(m,n,rdif,r,k), then:

$$N ( u im , vjn ) = N ( ui , vj ) * D ( m , n , rdif , k )$$

where the parameters in D(m,n,rdif,r,k) that determine D include, in addition to the DOI layers m and n, the crystal ring difference rdif=|u−v|, the radial index r(irad) of the target LOR, and the transaxial angular index k(iphi) of the target LOR. In some embodiments, each parameter in the DOI compensating coefficient for the NC factor can be obtained from the sinusoidal data corresponding to the target LOR, e.g., by looking up a table.

In the same PET system, the total number of crystal layers (i.e., the number of DOI layers) corresponding to detector crystals in different rings is the same. For example, the total number of the crystal layers can be defined by n_{DOI}.

After the gamma ray enters the crystal, a deposition probability among different DOI crystals is determined by an incident direction of the gamma ray, a thickness of the DOI layer, and/or a capability of other crystals to block the ray before the ray enters the DOI layer. Therefore, the determination of the DOI compensating coefficient for the NC factor (e.g., D(m,n,rdif,r,k)) is related to a direction of the target LOR relative to the crystal. The direction of the target LOR of the coincidence event relative to the crystal is determined based on the above described one or more parameters of the DOI compensating coefficient for the NC factor (e.g., (m,n,rdif,r,k)).

In this embodiment, the complexity of the correction coefficient D can be simplified by considering rotational and/or translational symmetries of the PET system.

Different target LORs with annular rotational symmetry can share one or more same parameters of the parameters of the DOI compensating coefficient. For example, if one target LOR is rotated from its original position for a certain angle around an axis of the rings in the system, the rotated one target LOR can be substantially the same in position as (that is, substantially overlapping with) another target LOR in the system, then we can regard the two target LORs as having annular rotational symmetry with each other.

For example, in an embodiment where the DOI compensating coefficient at least includes the above parameter of the transaxial angular index k, then different target LORs with annular rotational symmetry can share the same transaxial angular index k. The parameter k can be simplified as k=mod(iₚₕᵢ,n_{sym}_{ₚₕᵢ}) when the rotational symmetry is considered, where iₚₕᵢ refers to the transaxial angular index of the target LOR without considering the rotational symmetry, and n_{sym}_{ₚₕᵢ} is the number of symmetric angles of the target LORs. Exemplarily, the number of selectable values from which k can select to be (which corresponds to the selectable dimension of k) being nₒᵣᵢ_{ₚₕᵢ} when the rotational symmetry is not considered, can be simplified to nₒᵣᵢ_{ₚₕᵢ}/n_{sym}_{ₚₕᵢ} when the rotational symmetry is considered. For example, assuming that a PET detector has 1000 detector crystals in one ring, generally there would be 500 angles defined in a range of 0 to 180 degrees when defining the angle, i.e., there are 500 different angles in the range of 0 to 180 degrees. For different target LORs with annular rotational symmetry, the same coefficient can be used, therefore the input parameter is selected to be the angle coefficient k. Assuming that the detector crystal ring is designed to have a shape of a 50-sided polygon, with each side hosting 20 detectors, then the 0^{th} angle and the 20^{th} angle are rotationally symmetric with each other, that is, n_{sym}_{ₚₕᵢ}=25. In this case, only a coefficient k in a value range of 0 to 19 needs to be generated, and the others can be directly looked up through rotational symmetry. Without considering the symmetry, a value range of iₚₕᵢ is 0 to 499, while with considering the symmetry, the value range of iₚₕᵢ is 0 to 19. The selectable dimension of k is changed from its original 0-499 to 0-19, that is, the number of selectable values of k is changed from nₒᵣᵢ_{ₚₕᵢ}=500 when the rotational symmetry is not considered to

$$\frac{{n}_{o r {i}_{p h i}}}{{n}_{s y {m}_{p h i}}} = 500 / 25 = 20$$

when the rotational symmetry is considered, thus simplifying the data volume in the lookup table.

For LORs that are symmetrical in axial translation, they can share one or more same parameters of the parameters of the DOI compensating coefficient. If one target LOR is translated from its original position for a certain distance along the axis of the rings in the system, the translated one target LOR can be substantially the same in position as (that is, substantially overlapping with) another target LOR in the system, then we can regard the two target LORs as being symmetrical in axial translation with each other.

In an embodiment where the DOI compensating coefficient at least includes the above parameter of the crystal ring difference rdif, then different target LORs being symmetrical in axial translation with each other can share the same parameter of crystal ring difference rdif. For example, the target LOR of the coincidence event between the r1^{th}=0^{th} ring and the r2^{th}=1^{st} ring, and the target LOR of the coincidence event between the r3^{th}=1^{st} ring and the r4^{th}=2^{nd} ring in the axial direction, are symmetrical in axial translation with each other since r4−r3=r2−r1, so they can share the same crystal ring difference rdif, therefore the input parameter is selected to be the ring difference rdif.

The parameter dimension (i.e., the selectable dimension of each of the parameters, the total number of the parameters, what parameters are considered) of the DOI compensating coefficient for the NC factor (e.g., D(m,n,rdif,r,k)) of the target LOR relative to the initial LOR in the present disclosure is only exemplary, and should not be regarded as limiting the protection scope of the present disclosure. The parameter dimension of the DOI compensating coefficient for the NC factor (e.g., D(m,n,rdif,r,k)) is determined and simplified by using symmetry. If the symmetry is not used, then the parameter dimension can be increased, or the parameter dimension can as well be simplified under a tolerance to a certain degree of error, which should all be within the protection scope of the present disclosure.

In this embodiment, it is defined that an initial LOR of a coincidence event between a pair of coincident detector crystals, is divided into n_{DOI}×n_{DOI} target LORs under the DOI structure with n_{DOI} layers. The sum of the efficiency expectations of these target LORs is equal to the initial LOR, and the DOI compensating coefficient for the NC factor can be regarded as an inverse of the efficiency expectation of the target LOR, so it is defined as follows:

$${\sum }_{m = 1}^{{n}_{DOI}} {\sum }_{n = 1}^{{n}_{DOI}} \frac{1}{D ( m , n , r d i f , r , k )} = 1$$

The method for determining the DOI compensating coefficient for the NC factor (e.g., D(m,n,rdif,r,k)) includes analytical calculation based on crystal geometry, an attenuation coefficient, and an incidence direction of the ray.

For analytical calculation, two-dimensional analytical calculation is taken as an example below.

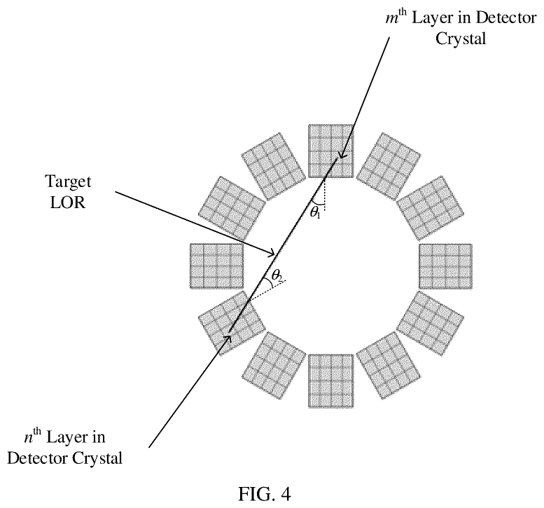

Assuming that a target LOR of the coincidence event is as shown in FIG. 4, the DOI information is divided along a depth direction of the crystal. The DOI information at one end of the coincidence event is the m^{th} layer, i.e., a deposition depth at one end of the coincidence event is the m^{th} layer, and the DOI information at the other end of the coincidence event is the n^{th} layer (counting from 0), i.e., a deposition depth at the other end of the coincidence event is the n^{th} layer. If the angles of the target LOR relative to the surface of the detector are θ₁ and θ₂, respectively, a DOI crystal has a total of n_{DOI} layers, and a thickness of a single crystal layer is d, then the detection efficiency of the target LOR in the crystal is related to a linear attenuation coefficient μ of the crystal. The DOI compensating coefficient for the NC factor of the target LOR corresponding to the m^{th} layer and the n^{th} layer can be approximated as:

$$D = \frac{( 1 - {e}^{- \frac{μ {n}_{DOI} d}{cos ( {θ}_{1} )}} ) ⋆ ( 1 - {e}^{- \frac{μ {n}_{DOI} d}{cos ( {θ}_{1} )}} )}{( {e}^{- \frac{μ ( m - 1 ) d}{cos ( {θ}_{1} )}} - {e}^{- \frac{μ md}{cos ( {θ}_{1} )}} ) ⋆ ( {e}^{- \frac{μ ( n - 1 ) d}{cos ( {θ}_{2} )}} - {e}^{- \frac{μ nd}{cos ( {θ}_{2} )}} )}$$

The DOI compensating coefficient for the NC factor of the target LOR relative to the initial LOR can be calculated by the above formula.

For the DOI crystal with DOI information corresponding to n_{DOI} layers, the number of LORs is n_{DOI}² times that of the crystal without the DOI information. For example, for a DOI crystal with 8 layers, the number of LORs is 64 times that of a crystal without the DOI information. The more layers there are, the greater the amount of calculation will be, which significantly increases the complexity and statistics of the correction.

In the correction method for a PET system of the present embodiment, based on the traditional initial NC factor of the initial LOR unrelated to the DOI information, the DOI compensating coefficient for the NC factor of the target LOR related to the DOI information relative to the initial LOR is introduced. Then the target NC factor corresponding to the target LOR is obtained through the product of the initial NC factor and the DOI compensating coefficient for the NC factor, thereby achieving the NC for the PET system. Without increasing the complexity and statistics of the correction, the accuracy of the reconstruction of image by the detector in the PET system with the DOI information is improved, and the maintainability of the PET system is significantly improved.

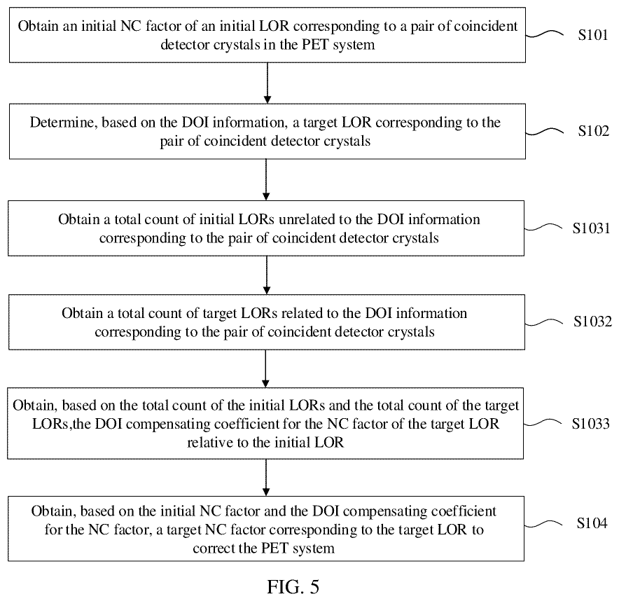

In some embodiments, as shown in FIG. 5, the above step S103 includes the following steps.

In step S1031, a total count of initial LORs unrelated to the DOI information corresponding to the pair of coincident detector crystals is obtained.

In step S1032, a total count of target LORs related to the DOI information corresponding to the pair of coincident detector crystals is obtained.

In step S1033, the DOI compensating coefficient for the NC factor of the target LOR relative to the initial LOR is obtained based on the total count of the initial LORs and the total count of the target LORs.

The method for determining the DOI compensating coefficient for the NC factor (e.g., D(m,n,rdif,r,k)) further includes Monte Carlo simulation and/or experimental measurement. Through the Monte Carlo simulation or standard phantom experiment, the count amount (total count) of the target LORs of the coincidence events with different DOI information corresponding to the pair of coincident detector crystals, and the total count of the initial LORs unrelated to the DOI information corresponding to the pair of coincident detector crystals are obtained. Further, based on the total count of the initial LORs and the total count of the target LORs, the DOI compensating coefficient for the NC factor of the target LOR relative to the initial LOR is determined.

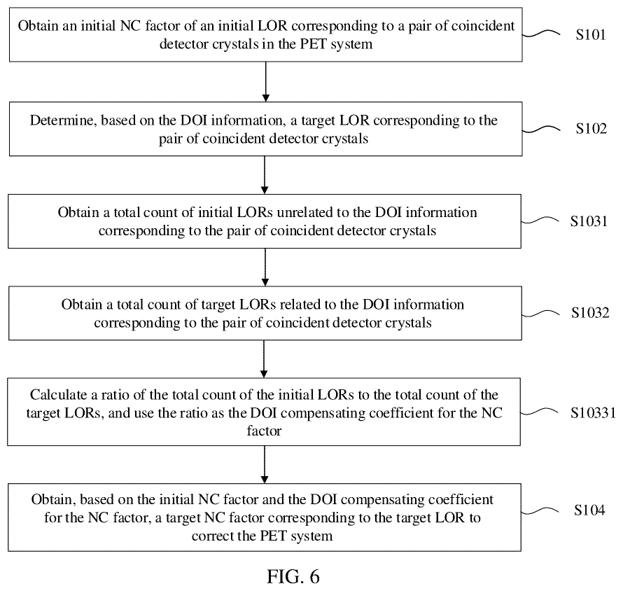

In some embodiments, as shown in FIG. 6, the above step S1033 includes the following step.

In step S10331, a ratio of the total count of the initial LORs to the total count of the target LORs is calculated, and the ratio is used as the DOI compensating coefficient for the NC factor.

Specifically, the ratio of the count amount of the initial LORs to the count amount of the target LORs of the coincidence events with different DOI information is obtained through the Monte Carlo simulation or the standard phantom experiment.

The formula for determining the DOI compensating coefficient for the NC factor based on the ratio of the count amount of the initial LORs to the count amount of the target LORs is represented as follows:

$$D ( m , n , r d i f , r , k ) = \frac{C ( rdif , r , k )}{C ( m , n , r d i f , r , k )}$$

where C(rdif,r,k) is the total count of the initial LORs unrelated to the DOI information corresponding to the pair of coincident detector crystals, and C(m,n,rdif,r,k) is the total count of the target LORs related to the DOI information corresponding to the pair of coincident detector crystals. Specifically, C(rdif,r,k) is the total count of the initial LORs unrelated to the DOI information with ring difference being rdif, radial index being r, and transaxial angular index being k, and C(m,n,rdif,r,k) is the count of the target LORs with crystal ring difference being rdif, radial index being r, transaxial angular index being k, DOI layer at one end being m, and DOI layer at the other end being n.

Since the NC is applied to a true coincidence event that has not been scattered or deflected and before entering the detector, a non-attenuation phantom that covers effective LORs in the entire space is generally used as a simulation phantom in the Monte Carlo simulation. The solutions that can be used include a full-space non-attenuation bucket source, a rotating surface source, a rotating rod source, or the like. In experimental measurement, in order to reduce scattering events as much as possible, the rotating rod source or the rotating surface source is generally used for measurement.

Generally, for the sake of convenience, DOI compensating coefficient for the NC factor (e.g., D(m,n,rdif,r,k)) is pre-stored list data, but DOI compensating coefficient for the NC factor (e.g., D(m,n,rdif,r,k)) calculated in real-time also falls within the protection scope of the present disclosure.

In the correction method for a PET system of the present embodiment, the DOI compensating coefficient for the NC factor of the target LOR related to the DOI information relative to the initial LOR can be quickly and accurately calculated. Based on the traditional initial NC factor of the initial LOR unrelated to the DOI information, the target NC factor corresponding to the target LOR is obtained through the product of the initial NC factor and the DOI compensating coefficient for the NC factor, thereby achieving the NC for the PET system. Without increasing the complexity and statistics of the correction, the accuracy of the reconstruction of image by the detector in the PET system with the DOI information is improved, and the maintainability of the PET system is significantly improved.

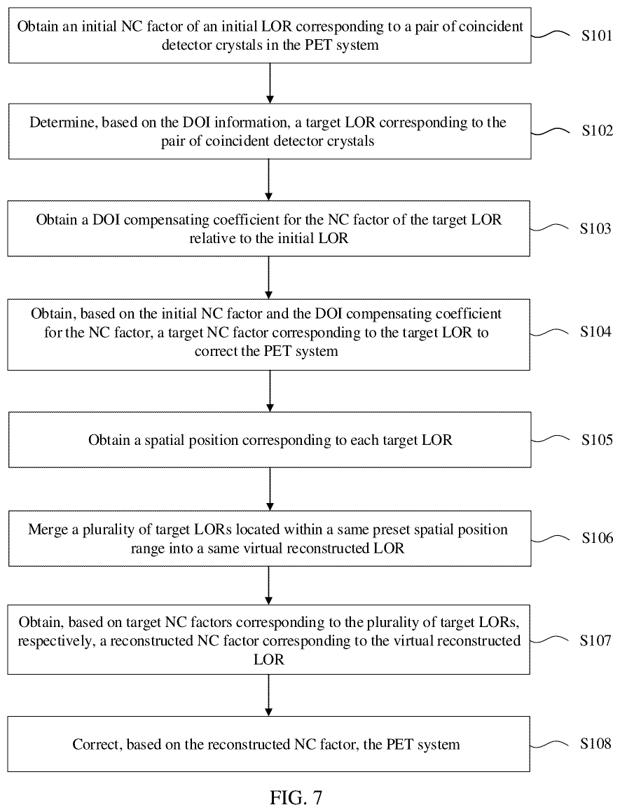

In an optional embodiment, as shown in FIG. 7, the correction method further includes the following steps.

In step S105, a spatial position corresponding to each target LOR is obtained.

In step S106, a plurality of target LORs located within a same preset spatial position range are merged into a same virtual reconstructed LOR.

In step S107, a reconstructed NC factor corresponding to the virtual reconstructed LOR is obtained based on target NC factors corresponding to the plurality of target LORs, respectively. In step S108, the PET system is corrected based on the reconstructed NC factor.

The plurality of target LORs located within the same preset spatial position range refer to a plurality of target LORs that are close in position in the space.

In PET image reconstruction, since there is an excessively large number of target LORs of the coincidence events between DOI layers, the reconstruction is required to be performed in the LOR dimension after data re-binning (rebin). In other words, the calculation is performed under an assumption that a plurality of target LORs that are close in position in the space (i.e., located within the same preset spatial position range) are merged into a same LOR (i.e., a virtual reconstructed LOR). In this case, the target NC factor of the virtual reconstructed LOR is represented by N_{rebin}, and the calculation formula of N_{rebin} is as follows:

$${N}_{r e b i n} = \frac{1}{{{{{Σ}_{k = 1}^{{n}_{rebin}} \frac{1}{{N}_{k}}}_{}}_{}}^{}}$$

where n_{rebin} is the total count (the number of lines) of all target LORs during the data re-binning process of the LORs, and Nₖ is the target NC factor for each target LOR, which is the N(uim,vjn) above-mentioned.

The LORs that are close in spatial position are regarded as one LOR. For example, hundreds of target LORs that are close to each other are approximately regarded as one virtual reconstructed LOR, thereby improving the reconstruction speed.

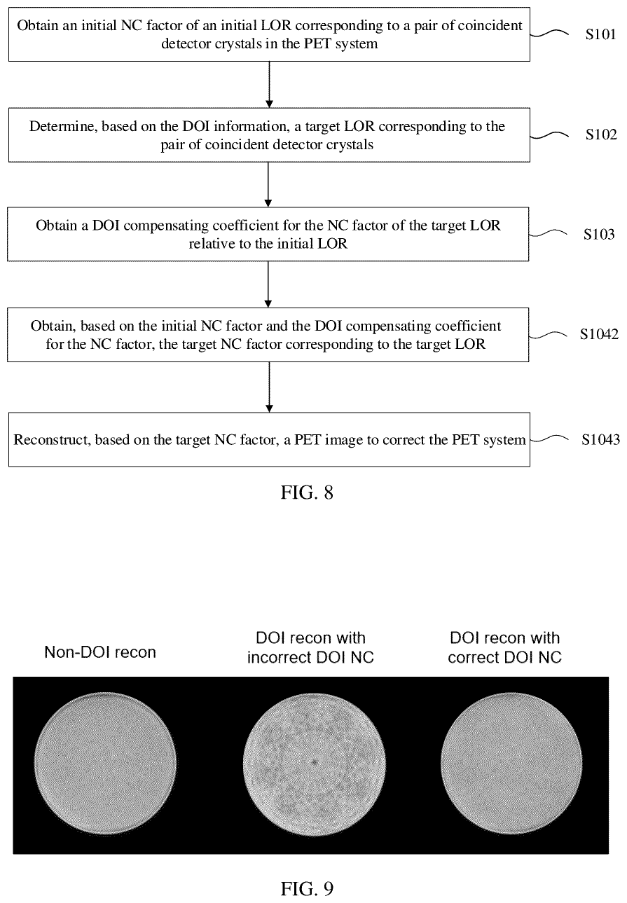

In an optional embodiment, as shown in FIG. 8, the above step S104 includes the following steps.

In step S1042, the target NC factor corresponding to the target LOR is obtained based on the initial NC factor and the DOI compensating coefficient for the NC factor.

In step S1043, a PET image is reconstructed based on the target NC factor, to correct the PET system.

After obtaining the target NC factor corresponding to the target LOR, the PET image is reconstructed based on the target NC factor, thereby achieving the correction for the PET system, thus improving the resolution of the reconstructed image.

FIG. 9 shows a comparison among a uniform water phantom reconstructed without using DOI information (i.e., Non-DOI recon), a uniform water phantom reconstructed using an incorrect DOI NC factor (i.e., DOI recon with incorrect DOI NC), and a uniform water phantom reconstructed using the correct target NC factor according to the present disclosure (i.e., DOI recon with correct DOI NC). From the comparison, it can be seen that the reconstructed water phantom image using the target NC factor of the present disclosure is highly uniform, thereby confirming the superiority of the technical solution of the present disclosure.

### Embodiment Two

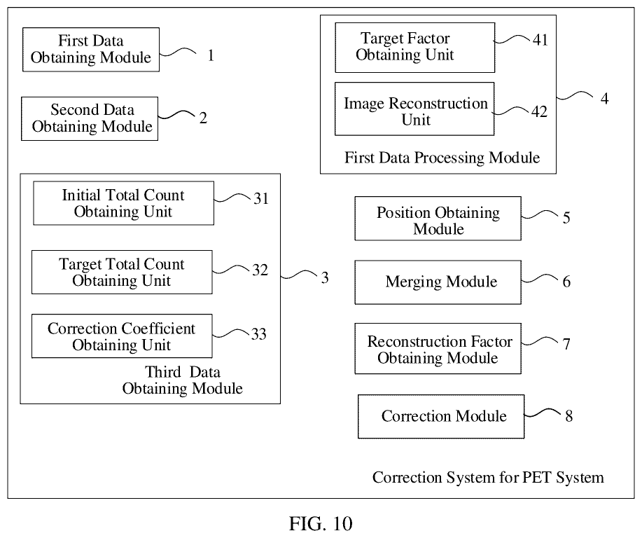

The present embodiment provides a correction system for a PET system, which is applied to a PET system including DOI information. As shown in FIG. 10, the correction system includes:
a first data obtaining module 1 configured to obtain an initial NC factor of an initial LOR corresponding to a pair of coincident detector crystals in the PET system; the initial LOR being unrelated to the DOI information;
a second data obtaining module 2 configured to determine, based on the DOI information, a target LOR corresponding to the pair of coincident detector crystals; the target LOR being related to the DOI information;
a third data obtaining module 3 configured to obtain a DOI compensating coefficient for the NC factor of the target LOR relative to the initial LOR; and
a first data processing module 4 configured to obtain, based on the initial NC factor and the DOI compensating coefficient for the NC factor, a target NC factor corresponding to the target LOR to correct the PET system.

In an optional embodiment, the correction system may alternatively include:
a fourth data obtaining module configured to determine, based on the DOI information, a target LOR corresponding to a pair of coincident detector crystals in the PET system; the target LOR being related to the DOI information;
a fifth data obtaining module configured to obtain an initial NC factor of an initial LOR corresponding to the pair of coincident detector crystals; the initial LOR being unrelated to the DOI information;
a sixth data obtaining module configured to obtain a DOI compensating coefficient for the NC factor of the target LOR relative to the initial LOR; and
a second data processing module configured to obtain, based on the initial NC factor and the DOI compensating coefficient for the NC factor, a target NC factor corresponding to the target LOR to correct the PET system.

It is to be noted that, the function of the fourth data obtaining module is similar to that of the second data obtaining module 2, the function of the fifth data obtaining module is similar to that of the first data obtaining module 1, the function of the sixth data obtaining module is similar to that of the third data obtaining module 3, and the function of the second data processing module is similar to that of the first data processing module 4. Therefore, the configurations of the fourth data obtaining module, the fifth data obtaining module, the sixth data obtaining module, and the second data processing module can be referred to the descriptions above and below for the configurations of the second data obtaining module 2, the first data obtaining module 1, the third data obtaining module 3, and the first data processing module 4, respectively.

In an optional embodiment, the first data obtaining module 1 is further configured to obtain, based on a preset NC method, the initial NC factor of the initial LOR corresponding to the pair of coincident detector crystals in the PET system.

The preset NC method includes but is not limited to a direct NC method and a component NC method.

In an optional embodiment, the first data processing module 4 is further configured to determine a product of the initial NC factor and the DOI compensating coefficient for the NC factor, and use the product as the target NC factor to correct the PET system.

In an optional implementation, the third data obtaining module 3 includes:
an initial total count obtaining unit 31 configured to obtain a total count of initial LORs unrelated to the DOI information corresponding to the pair of coincident detector crystals;
a target total count obtaining unit 32 configured to obtain a total count of target LORs related to the DOI information corresponding to the pair of coincident detector crystals; and
a correction coefficient obtaining unit 33 configured to obtain, based on the total count of the initial LORs and the total count of the target LORs, the DOI compensating coefficient for the NC factor of the target LOR relative to the initial LOR.

In an optional embodiment, the correction coefficient obtaining unit 33 is further configured to calculate a ratio of the total count of the initial LORs to the total count of the target LORs, and use the ratio as the DOI compensating coefficient for the NC factor.

In an optional embodiment, the correction system further includes:
a position obtaining module 5 configured to obtain a spatial position corresponding to each target LOR;
a merging module 6 configured to merge a plurality of target LORs located within a same preset spatial position range into a same virtual reconstructed LOR;
a reconstruction factor obtaining module 7 configured to obtain, based on target NC factors corresponding to the plurality of target LORs, respectively, a reconstructed NC factor corresponding to the virtual reconstructed LOR; and
a correction module 8 configured to correct, based on the reconstructed NC factor, the PET system.

In an optional embodiment, the first data processing module 4 includes:
a target factor obtaining unit 41 configured to obtain, based on the initial NC factor and the DOI compensating coefficient for the NC factor, the target NC factor corresponding to the target LOR; and
an image reconstruction unit 42 configured to reconstruct, based on the target NC factor, a PET image to correct the PET system.

The correction system for a PET system in this embodiment corresponds to the correction method for a PET system in Embodiment One, and the operating principle of the correction system for a PET system in this embodiment is the same as the operating principle of the correction method for a PET system in Embodiment One, which will not be repeated here.

In the correction system for a PET system of the present embodiment, based on the traditional initial NC factor of the initial LOR unrelated to the DOI information, the DOI compensating coefficient for the NC factor of the target LOR related to the DOI information relative to the initial LOR is introduced. Then the target NC factor corresponding to the target LOR is obtained through the initial NC factor and the DOI compensating coefficient for the NC factor, thereby achieving the NC for the PET system. Without increasing the complexity and statistics of the correction, the accuracy of the reconstruction of image by the detector in the PET system with the DOI information is improved, and the maintainability of the PET system is significantly improved.

### Embodiment Three

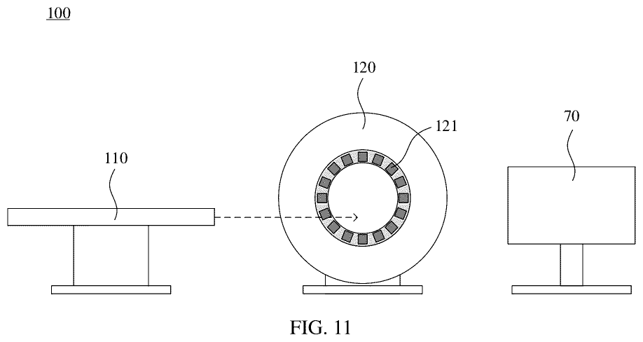

Referring to FIG. 11, the present embodiment provides a PET system 100. The PET system 100 includes the correction system for a PET system in Embodiment Two. The correction system for a PET system can be implemented by the electronic device 70 described in the subsequent embodiments.

The PET system 100 may further include other components, such as a bed 110, a housing 120, and detector crystals 121. The electronic device 70 may be provided inside the housing 120, may be provided outside the housing 120, or may be provided partially inside the housing 120 and partially outside the housing 120.

In the PET system of the present embodiment, on the basis of the correction system for a PET system in Embodiment Two, the DOI compensating coefficient for the NC factor of the target LOR related to the DOI information relative to the initial LOR is introduced based on the traditional initial NC factor of the initial LOR unrelated to the DOI information. Then the target NC factor corresponding to the target LOR is obtained through the initial NC factor and the DOI compensating coefficient for the NC factor, thereby achieving the NC for the PET system. Without increasing the complexity and statistics of the correction, the accuracy of the reconstruction of image by the detector in the PET system with the DOI information is improved, and the maintainability of the PET system is significantly improved.

### Embodiment Four

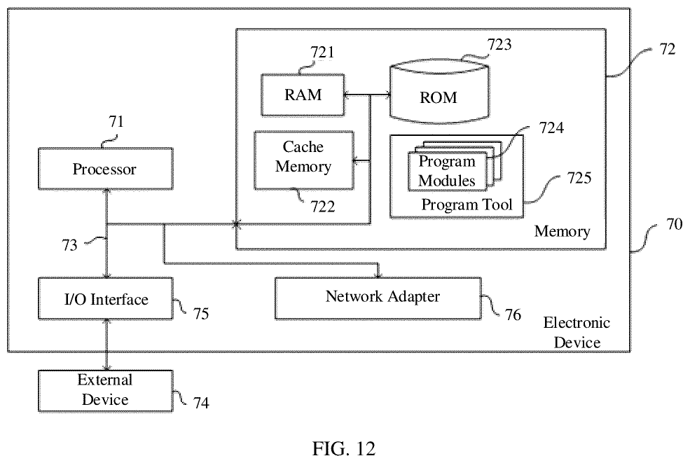

The present embodiment provides an electronic device 70. FIG. 12 is a schematic diagram illustrating a configuration of the electronic device 70 according to the present embodiment. The electronic device 70 includes a memory, a processor, and a computer program stored on the memory and capable of running on the processor. When the processor executes the computer program, the correction method for a PET system in the above Embodiment One is implemented. The electronic device 70 shown in FIG. 12 is merely an example, and should not be construed as any limitations to the functions and scope of use of the embodiments of the present disclosure. As shown in FIG. 12, the electronic device 70 may be in the form of a general-purpose computing device, for example, it may be a server device. The components of the electronic device 70 may include, but are not limited to: the at least one processor 71 mentioned above, the at least one memory 72 mentioned above, and a bus 73 connecting different system components (including the memory 72 and the processor 71).

The bus 73 includes a data bus, an address bus, and a control bus.

The memory 72 may include a transitory memory, such as a random-access memory (RAM) 721 and/or a cache memory 722, and may further include a read only memory (ROM) 723.

The memory 72 may further include a program tool 725 (or a utility tool) having a set of (at least one) program modules 724. The program modules 724 include, but is not limited to: an operating system, one or more application programs, other program modules and program data, each of which or a combination thereof may include the implementation of a network environment.

By running the computer program stored in the memory 72, the processor 71 executes various functional applications and data processing, such as the correction method for a PET system in the above Embodiment One.

The electronic device 70 may also communicate with one or more external devices 74. Such communication may be implemented via an input/output (I/O) interface 75. Further, the model-generated electronic device 70 may also communicate with one or more networks (e.g., a local area network (LAN), a wide area network (WAN) and/or a public network, such as the Internet) via a network adapter 76. As shown in FIG. 12, the network adapter 76 communicates with other modules of the electronic device 70 via the bus 73. It should be understood that although not shown in the figure, other hardware and/or software modules can be used by combined with the electronic device 70, including but not limited to: microcode, device drivers, redundant processors, external disk drive arrays, RAID (disk array) systems, tape drives, and data backup storage systems.

It should be noted that although several units/modules or sub-units/modules of the electronic device are mentioned in the above detailed description, such division is merely exemplary rather than compulsory. In fact, according to the embodiments of the present disclosure, the features and functions of two or more units/modules described above may be embodied in one unit/module. On the contrary, the features and functions of one unit/module described above may be further divided into multiple units/modules to be embodied.

### Embodiment Five

The present embodiment provides a computer-readable storage medium having a computer program stored therein. When the computer program is executed by a processor, the correction method for a PET system in the above Embodiment One is implemented.

The readable storage medium may include but is not limited to: a portable disk, a hard disk, a random-access memory, a read-only memory, an erasable programmable read-only memory, an optical storage device, a magnetic storage device or any suitable combination of the above.

In a possible implementation, the present disclosure may also be implemented in the form of a program product including a program code. When the program product runs on a terminal device, the program code is configured to cause the terminal device to implement the steps in the correction method for a PET system in the above Embodiment One.

The program code for executing the present disclosure may be written in any combination of one or more programming languages. The program code may be executed entirely or partly on a user device, be executed as a stand-alone software package, be executed partly on the user device and partly on a remote device, or entirely on the remote device.

Although specific embodiments of the present disclosure have been described above, a person skilled in the art will understand that these are only examples and the protection scope of the present disclosure is defined by the appended claims. The person skilled in the art may make various changes or modifications to these embodiments without departing from the principles and essence of the present disclosure, but these changes and modifications all fall within the protection scope of the present disclosure.

What is claimed is:

1. A correction method for a PET system, wherein the correction method is applied to a PET system comprising depth of interaction (DOI) information, and the correction method comprises:
obtaining an initial normalization correction (NC) factor of an initial line of response (LOR) corresponding to a pair of coincident detector crystals in the PET system; the initial LOR being unrelated to the DOI information;
determining, based on the DOI information, a target LOR corresponding to the pair of coincident detector crystals;
obtaining a DOI compensating coefficient for the NC factor of the target LOR relative to the initial LOR; and
obtaining, based on the initial NC factor and the DOI compensating coefficient for the NC factor, a target NC factor corresponding to the target LOR to correct the PET system.

2. The correction method according to claim 1, wherein the step of obtaining the initial NC factor of the initial LOR corresponding to the pair of coincident detector crystals in the PET system comprises:
obtaining, based on a preset NC method, the initial NC factor of the initial LOR corresponding to the pair of coincident detector crystals in the PET system.

3. The correction method according to claim 2, wherein the preset NC method includes a direct NC method and/or a component NC method.

4. The correction method according to claim 1, wherein parameters of the DOI compensating coefficient for the NC factor include one or more of: DOI information, crystal ring difference, radial index of the target LOR, or transaxial angular index of the target LOR.

5. The correction method according to claim 1, wherein values of parameters of the DOI compensating coefficient for the NC factor are obtained from sinusoidal data corresponding to the target LOR.

6. The correction method according to claim 4, wherein a parameter dimension of the DOI compensating coefficient for the NC factor is simplified by utilizing symmetry of the PET system.

7. The correction method according to claim 4, wherein the parameters of the DOI compensating coefficient for the NC factor include at least the transaxial angular index of the target LOR, and different target LORs with annular rotational symmetry share a same transaxial angular index.

8. The correction method according to claim 4, wherein the parameters of the DOI compensating coefficient for the NC factor include at least the crystal ring difference, and different target LORs with axial translation symmetry share a same crystal ring difference.

9. The correction method according to claim 1, wherein the step of obtaining, based on the initial NC factor and the DOI compensating coefficient for the NC factor, the target NC factor corresponding to the target LOR to correct the PET system comprises:
determining a product of the initial NC factor and the DOI compensating coefficient for the NC factor, and using the product as the target NC factor to correct the PET system.

10. The correction method according to claim 1, wherein the step of obtaining a DOI compensating coefficient for the NC factor of the target LOR relative to the initial LOR comprises:
calculating the DOI compensating coefficient for the NC factor of the target LOR relative to the initial LOR through an analytical calculation, the analytical calculation being based on at least one of crystal geometry, an attenuation coefficient, or an incidence direction of ray.

11. The correction method according to claim 1, wherein the step of obtaining the DOI compensating coefficient for the NC factor of the target LOR relative to the initial LOR comprises:
obtaining a total count of initial LORs unrelated to the DOI information corresponding to the pair of coincident detector crystals;
obtaining a total count of target LORs related to the DOI information corresponding to the pair of coincident detector crystals; and
obtaining, based on the total count of the initial LORs and the total count of the target LORS, the DOI compensating coefficient for the NC factor of the target LOR relative to the initial LOR.

12. The correction method according to claim 11, wherein the step of obtaining, based on the total count of the initial LORs and the total count of the target LORs, the DOI compensating coefficient for the NC factor of the target LOR relative to the initial LOR comprises:
calculating a ratio of the total count of the initial LORs to the total count of the target LORs, and using the ratio as the DOI compensating coefficient for the NC factor.

13. The correction method according to claim 1, wherein the correction method further comprises:
obtaining a spatial position corresponding to each target LOR;
merging a plurality of target LORs located within a same preset spatial position range into a same virtual reconstructed LOR;
obtaining, based on target NC factors corresponding to the plurality of target LORs, respectively, a reconstructed NC factor corresponding to the virtual reconstructed LOR; and
correcting, based on the reconstructed NC factor, the PET system.

14. The correction method according to claim 1, wherein the step of obtaining, based on the initial NC factor and the DOI compensating coefficient for the NC factor, the target NC factor corresponding to the target LOR to correct the PET system comprises:
obtaining, based on the initial NC factor and the DOI compensating coefficient for the NC factor, the target NC factor corresponding to the target LOR; and
reconstructing, based on the target NC factor, a PET image to correct the PET system.

15. A correction method for a PET system, wherein the correction method is applied to a PET system comprising depth of interaction (DOI) information, and the correction method comprises:
determining, based on DOI information, a target line of response (LOR) corresponding to a pair of coincident detector crystals in the PET system; the target LOR being related to the DOI information;
obtaining an initial normalization correction (NC) factor of an initial LOR corresponding to the pair of coincident detector crystals; the initial LOR being unrelated to the DOI information;
obtaining a DOI compensating coefficient for the NC factor of the target LOR relative to the initial LOR; and
obtaining, based on the initial NC factor and the DOI compensating coefficient for the NC factor, a target NC factor corresponding to the target LOR to correct the PET system.

16. A PET system, comprising an electronic device, a housing and detector crystals, wherein the electronic device is configured to perform the correction method for a PET system of claim 1.

17. The PET system according to claim 16, wherein the PET system comprises a plurality of rings, each of the rings has a certain number of detector crystals, and each of the detector crystals includes a plurality of layers.

18. The PET system according to claim 17, wherein detector crystals in different rings have a same total number of layers.

19. An electronic device comprising a memory and a processor, and a computer program executable by the processor is stored on the memory, wherein when the processor executes the computer program, the correction method for a PET system of claim 1 is implemented.

20. A non-transitory computer-readable storage medium having a computer program stored therein, wherein when the computer program is executed by a processor, the correction method for a PET system of claim 1 is implemented.
